(19) United States
(12) Patent Application Publication
(10) Pub. No.: US 2006/0177518 A1
(43) Pub. Date: Aug. 10, 2006

(54) Peracetic teat dip

(76) Inventors: Randal D. Stevenson, Romeoville, IL (US); Alejandro O. Dee, Roselle, IL (US)

Correspondence Address: SMITH LAW OFFICE, 440 SCIENCE DR., SUITE 302, MADISON, WI, 53711, US

(21) Appl. No.: 11/051,501

(22) Filed: Feb. 4, 2005

(51) Int. Cl. A61K 33/40; A61K 31/19; A61K 31/17; A61K 31/045

(52) U.S. Cl. 424/616; 514/557; 514/738; 514/588

(57) ABSTRACT

A teat dip formula is disclosed for use on dairy animals with the objective of reducing or preventing mastitis. The teat dip formula preferably includes up to about 5000 ppm of peracetic acid in solution made in situ by mixing acetic acid and hydrogen peroxide. Other constituents of the formula may include effective amounts of a skin conditioning agent or agents, a surfactant or surfactants and urea. A method of preparing and applying the formula is disclosed.

### FIELD OF THE INVENTION

The present invention is related to formulations and methods directed to the prevention of new intra-mammary infections in dairy cows. More particularly, the present invention is related to a teat dip formulation and method. Even more specifically, the present invention includes a method for the formulation of an in situ reaction of acetic acid with hydrogen peroxide in a teat dip formulation.

### BACKGROUND OF THE INVENTION

The treatment and prevention of mastitis in dairy cows continues to be of primary importance to the dairy industry. Mastitis is caused by an infection of the milk-producing mammary glands by a broad spectrum of pathogenic microorganisms. These could include such organisms as Staphylococcus aureus, Streptococcus agalactiae, Escherichia coli, Klebsiella pneumoniae and Mycoplasma bovis. In particular, when the milk-producing glands and surrounding tissues in the udder become infected, the tissues may become inflamed with cellular infiltrates and associated toxic substances.

The cellular infiltrates and associated toxins, along with the infecting organisms themselves, can cause a dramatic reduction in the quality of milk produced by the animal. The infiltrates, toxins, and organisms can also affect the quantity of milk produced by the animal. Occasionally, the infection can spread systemically to other organ and tissue sites via the blood or lymphatic systems. The spreading infection can, in extreme cases, seriously debilitate or kill the infected animal.

The most common method used to combat the problem involves treating the infected animals with antibiotics. In some cases, where the disease is chronic or the animal seriously debilitated, the animal may be permanently removed or “culled” from the herd. Antibiotics are usually administered directly into the mammary gland via the teat orifice but can also be administered systemically from other body sites.

A secondary problem of antibiotic treatment is the potential for antibiotic residue in the treated animals and their milk products. Antibiotic-contaminated milk cannot be sold within many nations, which can constitute a loss of income to the dairy producer. Overuse of antibiotics can promote the development of resistant strains of mastitis-causing pathogens, which are then more difficult and costly to control. Additionally, public opposition over the use of antibiotics and the presence of antibiotics residues in meat and milk products has severely limited their market.

As an alternative to treatment with antibiotics after infection, products have been designed to prevent mastitis by killing the pathogenic organisms that might otherwise infect and invade the teat and udder tissues before the organisms enter the teat canal. Proactive topical antiseptics commonly known as a teat (or udder) dips, washes, foams, sprays, or wipes, for example are applied to the teat and udder area of the dairy cow or other milk-producing animal before and/or after milking as part of a process of general dairy hygiene. Teat dips are intended to kill or reduce in number the mastitis-causing microorganisms on the surface of the teat before the microorganisms have had a chance to migrate or be propelled (during milking) into the teat canal, or to enter the teat via injuries or lesions. The various methods for applying a teat sanitation formulation are well known, and include dipping, spraying, foaming, wiping, and so on.

Although the wide-spread use of topical antiseptics in the last 30 years has greatly decreased the incidence of mastitis, many of the products presently used as teat dips, washes, sprays or wipes having broad-spectrum chemical germicides such as chlorinated compounds, iodophors or chlorhexidines are known to irritate the animals'skin. Irritation is even more likely when a cow is subjected to repeated applications of the product, two or three times a day, before and/or after milking, and over a period of years. Additionally, there is concern among regulatory agencies, such as the U.S. Food and Drug Administration, about the presence of germicide residues, such as iodine or chlorhexidine, in milk products.

Chemical germicides such as chlorine, iodine, and chlorhexidine compounds also lack a high degree of stability. These chemical germicides can become inactivated over time as a result of the instability, or can become inactivated by substances (such as water or organic materials), which may contaminate, react with or dilute the germicide after it has been applied to the teat.

One suggested substitute for the chlorinated compounds, iodophors and chlorhexidines presently used as teat dips, are the fatty acids and their derivatives. The antimicrobial or germicidal properties of short to medium-chain fatty acids (C₆ to C₁₄) and their derivatives (such as esters) have been known for some time. See U.S. Pat. No. 6,699,907 to Dee et al., U.S. Pat. No. 4,406,884 to Fawzi and U.S. Pat. No. 5,208,257 to Kabara; Viegas, et al., Inhibition of Yeast Growth by octanoic and Decanoic Acids Produced during Ethanolic Fermentation, Applied and Environmental Microbiology, January 1989; J. J. Kabara, Toxicological, Bactericidal and Fungicidal Properties of Fatty Acids and Some Derivatives, Journal of American Oil Chemists'Society, November 1979; J. Fay and R. Farias, Inhibitory Action of a Non-Metabolizable Fatty Acid on the Growth of Escherichia coli: Role of Metabolism and Outer Membrane Integrity, Journal of Bacteriology, December 1977; and J. J. Kabara, Antimicrobial Lipids: Natural and Synthetic Fatty Acids and Monoglycerides, Lipids, March 1977. Fatty acids have been included in the class of lipophilic weak acids, which are generally considered to be an important class of antimicrobial agents. See Thomas R. Comer, Synergism in the Inhibition of Bacillus subtilin by Combinations of Lipophilic Weak Acids and Fatty Alcohols, Journal of Antimicrobial Agents and Chemotherapy, pp. 1082-85 (June 1981).

Highly bactericidal and undiluted fatty acids are irritating to the skin and they may even be corrosive to dairy equipment. Fortunately, it has been found that dilute concentrations of fatty acids have antimicrobial efficacy. Hence, a significant amount of work has been done to prepare antimicrobial compositions using a fatty acid diluted, for example, with water.

Preparing such a composition diluted with water is complicated because short to medium-chain fatty acids are, at best, only slightly soluble in water. One solution to the relative insolubility of fatty acids has been to add hydrotropes to compositions containing low concentrations (0.1% to 5.0% by weight of composition) of a mixture of fatty acids (C₆ to C₁₂) to solubilize the fatty acids. In such a composition, the shorter-chained fatty acids (C₆ to C₉) may actually assist the action of the hydrotrope by helping to solubilize the longer species, and thereby improve the longer species'antimicrobial efficacy. See U.S. Pat. No. 4,404,040 to Wang, et al.

To maintain the antimicrobial action of the fatty acids in solution with water, however, the pH of the composition must be sufficiently low (below about 4.0) to allow the acids to remain in their active free acid form. A strong organic or inorganic acid must be added to lower the pH so that the fatty acid can remain in its active form.

Alternatively, U.S. Pat. No. 4,406,884 to Fawzi et al. teaches away from solubilizing the fatty acids in water, and instead, teaches that the antimicrobial efficacy of the fatty acids may be enhanced by supersaturating the aqueous phase of an aqueous lotion or gel with low concentrations of a mixture of short and medium-chain fatty acids. According to Fawzi et al., the supersaturated aqueous phase combined with the lipophilicity of the fatty acids provides the increased antimicrobial action, without resort to either a hydrotrope or solubilizer to maintain the fatty acids in solution with the water.

Surfactant liquids and applicators are well known for use as bovine teat dips, and typical publications concerning these applicators are found in U.S. Pat. Nos. 3,713,423 and 4,305,346. Publications concerning bovine teat dips include U.S. Pat. Nos. 5,534,266 and 5,720,984 the latter patent disclosing a non-ionic, laureth (11-16) carboxylic acid surfactant teat dip and hand foam, which is highly suitable for use in this invention. Publications concerning bovine teat dip formulations include U.S. Pat. Nos. 3,728,449; 4,012,504; 4,049,830; 4,759,931; 5,641,498; 5,368,868; 5,534,266; 5,616,348; 5,651,977; and 5,720,984. Polyethenoxy detergents and I₂are disclosed in an article by Benjamin Carroll in the Journal of Bacteriology, 69: 413-417, (1955). A PVP surfactant for a teat dips is also suitable, and so is one sold by Norman Fox & Co. under the trade name of NORFOX N-P9, and listed in “McCutcheon's Emulsifiers and Detergents”, 1989 (incorporated by reference herein) specifically for use with iodophors. U.S. Pat. No. 5,616,348, supra, discloses a polyethoxylated polyoxypropylene block copolymer (Poloxamer) and iodine, which is suitable as a bovine teat dip.

So, it can be seen that an effective, safe, and simple method and composition for treating bovine teat tissue and the like is an important, yet elusive goal in the industry. It would be highly desirable, therefore, to provide a teat dip formulation that provides an effective, safe, and simple reduction or elimination of a broad spectrum of mastitis causing bacteria, and that is also sufficiently stable and safe to mix and use. The demand for such a formulation is met by the present invention.

### SUMMARY OF THE INVENTION

Peracetic (also referred to as peroxyacetic acid) has been found to be an effective antimicrobial in a variety of applications, including when used as a topical anti-microbial. Peracetic acid, as a strong oxidizer, is very reactive at high concentrations and potentially explosive. It is also hard to handle due to its high degree of irritation to the skin and respiratory tract of humans as well as animals. One solution is to formulate a teat dip that produces peracetic acid in a small enough quantity to be safe to handle and not be irritating while maintaining considerable bactericidal efficacy. It is also desirable to maintain this concentration of peracetic acid in equilibrium over an extended period of time to produce an acceptable shelf-life. The present invention achieves these goals through the continual and controlled production of peracetic acid via an in situ reaction.

In one preferred embodiment of the present invention, a teat dip formula is provided for treating dairy animals with the objective of reducing the amount of mastitis-causing organisms. The teat dip formula includes up to about 5000 ppm of peracetic acid in solution, and preferably has at least 20 ppm of acetic acid, with 200 ppm being a more preferred lower end of the concentration range.

In other embodiments of the present invention, the solution may be aqueous. The composition may include skin conditioning agents in a concentration of up to about seventy-five percent (75%) by weight of the composition. The skin-conditioning agents may be moisturizers and it may contain glycerin, sorbitol, propane diol, and/or lanolin. The composition may further include about 0.1% to 8.0% by weight of a surfactant. The composition may further include up to about 1.0% by weight of urea.

In yet another embodiment of the present invention, a method is provided of reducing mastitis in a dairy animal, including preparing a teat dip composition, comprising up to about 5000 ppm of peracetic acid in an aqueous solution and applying the teat dip composition topically to the teats of the animal. In other embodiments of the present invention, the application may be performed before and/or after milking of the animal.

In yet another embodiment of the present invention, a method is provided for preparing a teat dip composition, including combining about 0.1% to 5.0% acetic acid with 0. 1% to 5.0% hydrogen peroxide with the balance of the formulation being water where peracetic acid is formed in situ as an aqueous solution. In other embodiments, the composition is mixed in situ to enhance efficacy while maintaining stability.

In other embodiments of the present invention, the method may further include adding up to about 75% glycerin, sorbitol, propane diol, and/or lanolin. The method may further include adding about 0.1% to 8.0% of a surfactant to the solution and/or adding up to 1.0% urea to the solution.

In still other embodiments of the present invention, the method may further include up to 1.0% of a thickening agent. This thickening agent may consist of polyvinyl pyrollidone, xanthan gum, guar gum, clay, methylcellulose, hydroxypropylcellulose, hydroxypropylmethylcellulose, carboxymethylcellulose, anionic carboxyvinyl polymers, or hydroxymethylcellulose, or combinations thereof.

The embodiments of the teat dip formula described herein overcome many of the disadvantages of prior teat dip formulas in a unique formulation, which is both safe to use and effective.

### DISCLOSURE OF THE PREFERRED EMBODIMENTS OF THE INVENTION

The present invention relates to a teat dip composition, a method of forming the composition, and a method for reducing or preventing mastitis in dairy animals. In a preferred embodiment of the invention, a stable solution of peracetic acid in an effective concentration is prepared by combining in water, a stoicheiometric amount of acetic acid and hydrogen peroxide. The concentration of peracetic acid formed is preferably in the range of up to about 5000 ppm. Peracetic acid concentrations of as little as 20 ppm will be present shortly after mixing the acetic acid and hydrogen peroxide, and should be considered within the scope of the present invention. The concentration of peracetic acid may vary; over time as ambient conditions change; and relative with concentrations of other ingredients in the formulation.

### EXAMPLE

It has been determined that a teat dip formula including peracetic acid can be produced via an in situ chemical reaction of acetic acid and hydrogen peroxide. The quantity of peracetic acid produced in this manner is safe, stable, and effective for reducing new intra-mammary infections of dairy animals.

It has also been determined that the quantity of peracetic acid can be produced stoicheiometrically by controlling the reactants, acetic acid and hydrogen peroxide, in accordance with the chemical formulation below:
CH₃COOH+H₂O₂<=>CH₃COOOH+H₂O

Exemplary results of the above stoichiometry are illustrated by one preferred embodiment of the present invention detailed in Table 1:

TABLE 1

| | PERCENTAGE |
|---|---|
| INGREDIENT | BY WEIGHT |
| WATER | 90.80 |
| ACETIC ACID | 1.60 |
| HYDROGEN PEROXIDE, 35% | 3.00 |
| SKIN CONDITIONING AGENT | 5.00 |
| SURFACTANT | 0.25 |
| UREA | 0.05 |

The peracetic acid concentrations resulting from the composition of Table 1 will stabilize in a range of about 800 ppm to about 1200 ppm over a four month period under laboratory conditions. Of course, concentrations will vary depending upon ambient conditions, storage time, and skin conditioning concentrations, for example. Peracetic acid concentrations in the ranges recited herein can be achieved to obtain a safe, stable, and non-irritating teat dip composition.

The skin conditioning agents include, for example, moisturizers and barriers. Moisturizers or humectants are additives that attract moisture to the outer layers of skin to keep it moist and supple. The preferred skin conditioning agent is a glycerin moisturizer (also referred to as glycerol). Other moisturizers include propylene glycol, sorbitol and aloe, for example. Barriers prevent the loss of moisture already present in the skin, e.g., lanolin or lanolin-derivatives, petrolatum, and mineral oil. Other skin conditioning agents contemplated by the invention include additives, such as vitamins, anti-oxidants and other skin health compounds.

Surfactants in the composition may include one or more of the following types of non-ionic surfactants: nonylphenol ethoxylates, alcohol ethoxylates, alcohol alkylates, sorbitan ester ethoxylates, ethoxylated alkyl-polyglucosides, alkyl ether carboxylates, and ethylene oxide-propylene oxide copolymers. It is contemplated that other surfactants may be used in combination with the present invention as would be understood by those with skill in the art.

Testing in the field has confirmed that the above composition is stable at the range of temperatures generally experienced on dairy farms. In-vitro lab testing for skin irritation, using the “Irritection”™ Assay System, has demonstrated that the above composition in non-irritating. Significant bactericidal efficacy has been confirmed with laboratory tests as well as controlled field trials done at a university.

While generically referred to as a teat dip, it will be understood that the compositions disclosed herein may be sprayed, foamed, or wiped, for example, or applied by any known suitable method. Furthermore, the composition formed may be applied pre-milking and/or post-milking. In addition, it will be understood that the present invention is not limited to the composition of the preferred embodiment, but may contain other constituents for providing antimicrobial action and/or skin conditioning and related benefits.

The invention has been described with reference to preferred embodiments. Modifications and alterations will occur to others upon a reading and understanding of the detailed description. It is intended that the invention be construed as including all such modifications and alterations.

1. A teat dip composition for use with dairy animals, comprising up to about 5000 ppm of peracetic acid in solution.

2. The teat dip composition of claim 1, wherein the peracetic acid is present in a range from at least about 20 ppm.

3. The composition of claim 1, wherein said solution is aqueous.

4. The composition of claim 1, and further comprising up to 75% by weight of skin-conditioning agents.

5. The composition of claim 4, wherein said skin-conditioning agents comprises a moisturizer.

6. The composition of claim 4, wherein said skin-conditioning agent is glycerin.

7. The composition of claim 1, and further from about 0.2% to about 2.0% of surfactant by weight of a composition.

8. The composition of claim 1, and further including up to about 1.0% by weight of urea.

9. The composition of claim 1, wherein the peracetic acid is formed in situ by mixing hydrogen peroxide and acetic acid.

10. The composition of claim 9, wherein there is from about 0.1% to about 5.0% acetic acid and from about 0.1% to about 5.0% hydrogen peroxide by weight of the composition.

11. The composition of claim 1, and further including up to about 1.0% of a thickening agent selected from the group consisting of: polyvinyl pyrollidone, xanthan gum, guar gum, clay, methylcellulose, hydroxypropylcellulose, hydroxypropylmethylcellulose, carboxymethylcellulose, anionic carboxyvinyl polymers, hydroxymethylcellulose, and combinations thereof.

12. A method of preparing a teat dip composition, comprising the steps of:
mixing up to about 5000 ppm of peracetic acid with water.

13. The method of claim 12, wherein at least about 20 ppm of peracetic acid is mixed with water.

14. The method of claim 13, wherein the peracetic acid is produced via an in situ chemical reaction of acetic acid and hydrogen peroxide.

15. The method of claim 14, wherein the acetic acid is present in an amount of from about 0.1% to about 5.0% and the hydrogen peroxide is present in an amount of from about 0.1% to about 5.0% with the balance of the formulation being made by adding water to form an aqueous solution of peracetic acid.

16. The method of claim 15, wherein the aqueous solution of peracetic acid reaches a stable level of up to about 5000 ppm peracetic acid.

17. The method of claim 15, and further including the step of:
adding up to about 75% of glycerin by weight of the composition.

18. The method of claim 15, and further including the step of:
adding about 0.2% to about 2.0% of a surfactant by weight of the composition.

19. The method of claim 15, and further including the step of:
adding up to about 1.0% urea by weight of the composition.

20. The method of claim 15, and further including the step of adding up to 1.0% of a thickening agent selected from the group consisting of: polyvinyl pyrollidone, xanthan gum, guar gum, clay, methylcellulose, hydroxypropylcellulose, hydroxypropylmethylcellulose, carboxymethylcellulose, anionic carboxyvinyl polymers, hydroxymethylcellulose, and combinations thereof.

21. A method of reducing mastitis in a dairy animal, comprising the step of topically applying an antimicrobial composition to the teats of the animal, the composition comprising a solution of up to about 5000 ppm of peracetic acid.

22. The method of claim 21, wherein the composition is prepared by:
forming the peracetic acid by mixing acetic acid and hydrogen peroxide in situ.

23. The method of claim 21, wherein the composition comprises at least about 20 ppm of acetic acid.

24. The method according to claim 21, wherein the acetic acid is present in an amount of about 1.0% to about 5.0% by weight of the composition.

25. The method according to claim 21, wherein the hydrogen peroxide is present in an amount of about 0.1% to about 5.0% by weight of the composition.

26. The method according to claim 21, wherein the composition comprises a skin conditioning agent in an amount of up to about 75% by weight of the composition.

27. The method according to claim 21, wherein the composition comprises a surfactant in an amount of from about 0.2% to about 2.0% by weight of the composition.

28. The method according to claim 21, wherein the composition comprises urea in an amount of up to about 1.0% by weight of the composition.

29. The method of claim 21, wherein the applying step is performed before milking of the animal.

30. The method of claim 21, wherein the applying step is performed after milking of the animal.

31. The composition of claim 1, and further including up to about 1.0% by weight of a thickening agent.

32. The method of claim 12, and further comprising the step of:
mixing up to about 1.0% by weight of a thickening agent with the water and peracetic acid.

33. The method of claim 21, wherein the composition further comprises:
up to about 1.0% by weight of a thickening agent.
